(19) United States
(12) Patent Application Publication
(10) Pub. No.: US 2025/0332399 A1
(43) Pub. Date: Oct. 30, 2025

(54) Medical Valve

(71) Applicant: GUERBET, Villepinte (FR)

(72) Inventor: Ludovic ALLARD, Millery (FR)

(21) Appl. No.: 18/855,959

(22) PCT Filed: Apr. 7, 2023
(86) PCT No.: PCT/EP2023/059314

§ 371 Date: Oct. 10, 2024

(30) Foreign Application Priority Data
Apr. 11, 2022 (FR) 2203287

(51) Int. Cl. A61M 39/22

(52) U.S. Cl. CPC A61M 39/223; A61M 2039/229

(57) ABSTRACT

The invention relates to a valve comprising a body provided with at least three ports and a plug (20). The plug comprises a tubular barrel (21), the outer surface (22C) of a tubular wall (22) of which is complementary to an internal volume of the body in such a way that the barrel is sealingly arranged therein while being capable of being rotated about a central axis (Z21) of the barrel. The plug comprises a channel (25) connecting the ports according to the angular position of the barrel. The channel passes through the tubular wall, from its outer surface to its inner surface (22D), and extends inside the barrel by joining together at least two distinct openings each located in the same portion of the tubular wall extending over an angle of no more than 180°. The tubular wall has, on either axial side of the channel, a greater internal thickness in this portion than in the rest of the tubular wall.

The present invention relates to a medical valve.

The invention relates notably, although not exclusively, to medical valves used in interventional radiology.

The invention is concerned with medical valves comprising a hollow body, which is provided with at least three ports and inside which is rotatably mounted a plug which controls the establishment of communication, through it, between at least two of the ports of the valve body. The vast majority of valves of this type present on the market are made for uses involving pressures lower than 3 bar, for example for perfusion purposes. Be that as it may, for certain uses, the medical valves used are subjected to far higher pressures, potentially exceeding 50 bar and reaching or even exceeding 100 bar. Such is notably the case in interventional radiology, for example for performing ultra-selective trans-arterial chemoembolization, commonly referred to as cTACE, where micro-catheters are used to inject, as close as possible to tumors, and by passing through small-sized blood vessels, viscous mixtures containing aqueous solutions and oils, which requires these mixtures to be driven using pressures that can reach the aforementioned high values. WO 2016/166346 discloses an example of a medical valve suited to this context of use.

The higher the usage pressures, the greater the risk of the medical valves leaking, particularly at the interface, which is normally fluidtight, between the body and the plug of the valve. In order to improve this sealing, one widely held opinion is to increase the contact interference, notably the degree of force-fitting, between the body and the plug. However, this approach leads to a considerable increase in the torque needed for the rotary movement of the plug, rendering the valve somewhat impractical to use.

The object of the present invention is to propose an improved medical valve the resistance to leakage of which is improved, but without significantly increasing constraints on the use of the valve, notably the torque needed for the rotary movement of the plug.

To this end, the subject of the invention is a medical valve as defined in claim 1.

Contrary to the technical preconception that resistance to leakage can be effectively combated only by increasing the outside diameter of the plug in order to increase the contact interference between the valve body and the barrel of the valve plug, the inventors have demonstrated that it was more advantageous not to increase the contact interference but rather to maintain the interference while increasing the thickness of the barrel by reducing the inside diameter of the barrel. In addition, rather than increasing the thickness of the barrel in this way over the entire periphery of the barrel, the inventors have also demonstrated that it was far more advantageous to thicken the barrel internally only in the portion of the tubular wall of this barrel at the site of the openings via which the passage inside the plug opens onto the exterior lateral surface of this tubular wall. That leads to an off-centering of the inside diameter of the barrel with respect to the outside diameter thereof. The invention thus makes provision for the barrel to be internally thickened, and therefore rendered stiffer, exclusively in the aforementioned portion thereof, which is where the greatest pressure is applied when the medical valve is in use, while the diametrically opposite portion of the barrel is not thickened, allowing the pressure field to be distributed more uniformly and minimizing the risk of leakage to the outside of the interface between the plug and the body of the valve. By virtue of the invention, the medical valve withstands high pressures, notably greater than 65 bar and potentially as high as 80 bar, or even 100 bar, without leaks appearing, while at the same time still remaining easy to operate. In practice, as detailed hereinafter, the localized internal thickening of the barrel may exhibit various advantageous arrangements and/or be combined with enhanced thickness of the passage and with the reinforcing of a partition that holds this passage, notably with a view to further improving the sealing performance of the medical valve according to the invention.

Additional advantageous features of the medical valve according to the invention are specified in the other claims.

The invention will be better understood from reading the following description, given solely by way of example and made with reference to the drawings in which:

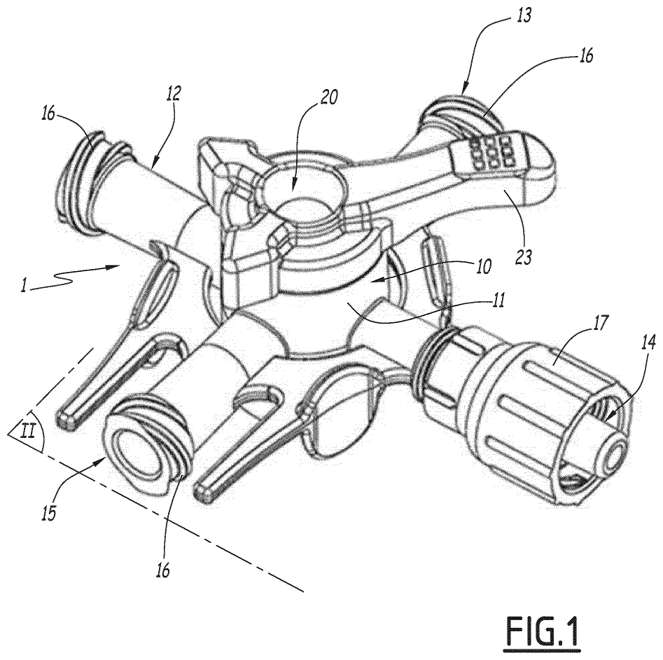

FIG. 1 is a perspective view of a medical valve according to the invention;

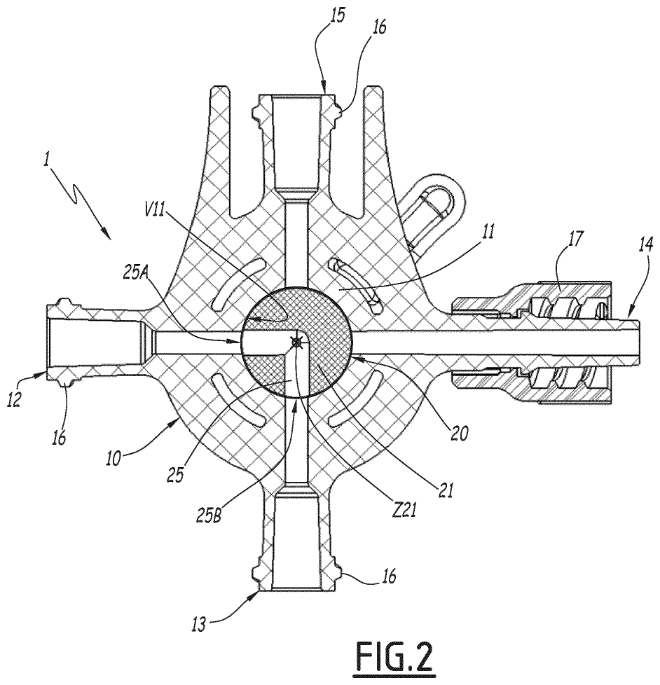

FIG. 2 is a cross section on the plane II of FIG. 1;

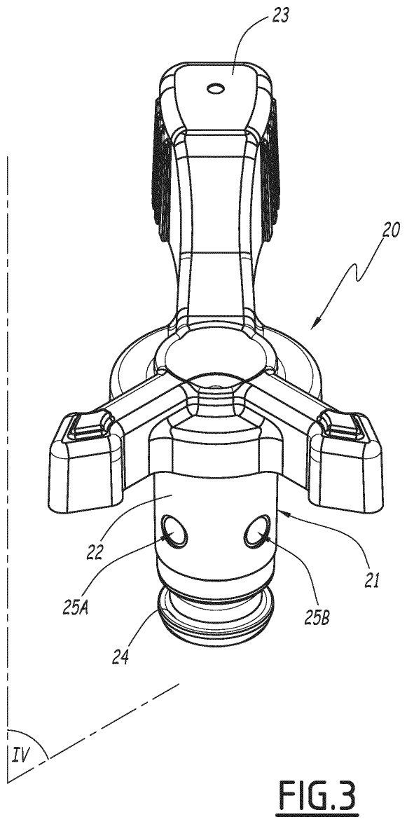

FIG. 3 is a perspective view of a first embodiment of a plug of the medical valve of FIGS. 1 and 2;

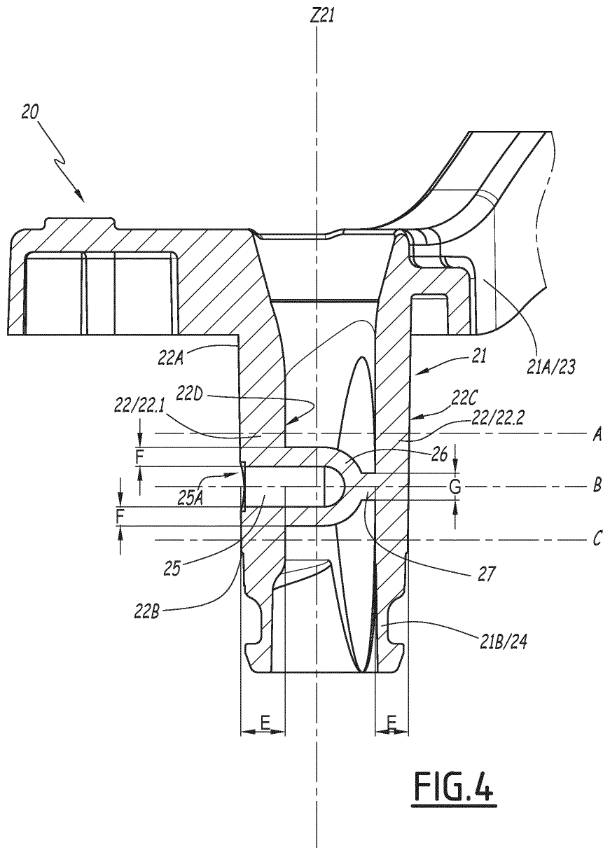

FIG. 4 is a cross section on the plane IV of FIG. 3;

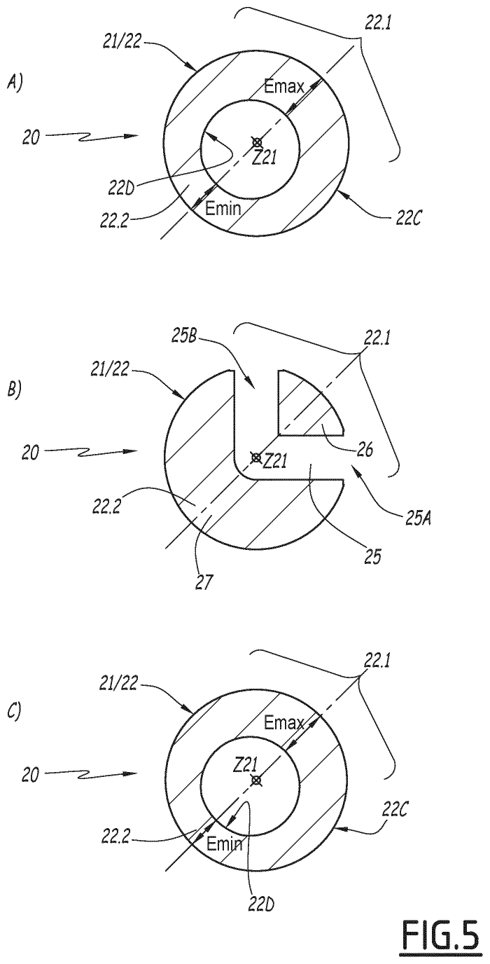

FIG. 5 is a grouping of inserts A), B) and C) which respectively correspond to sections on the lines A, B and C of FIG. 4; and

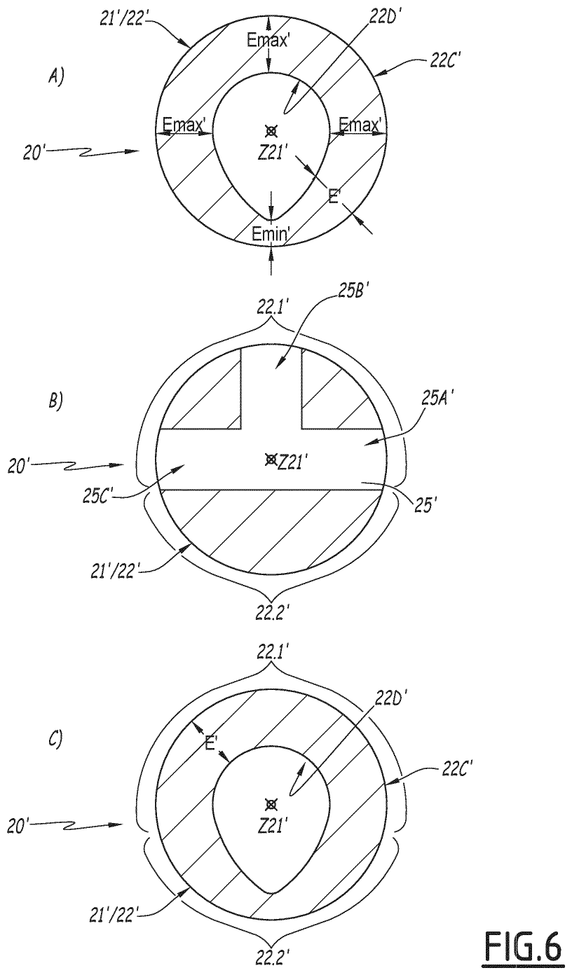

FIG. 6 is a view similar to FIG. 5, illustrating a second embodiment of the plug.

FIGS. 1 and 2 depict a medical valve 1. This medical valve 1 is notably, although not exclusively, able to be used in interventional radiology, notably to perform trans-arterial chemoembolization, commonly referred to as cTACE, possibly ultra-selectively, which is to say performed at the distal-most part of the vascularization of a tumor. The medical valve 1 can notably be used in the context and in accordance with the method which are detailed in WO2016/166346 to which the reader may refer for details on the corresponding specifics of the medical valve 1.

In any case, the medical valve 1 comprises a body 10 and a plug 20.

The body 10 comprises a tubular barrel 11 which, as shown in FIG. 2, defines an internal volume V11 inside which is housed the plug 20, as detailed later.

The body 10 also comprises four ports 12, 13, 14 and 15 each of which extends from the barrel 11 away from the internal volume V11 and which here are uniformly distributed about the exterior periphery of the barrel 11. Each port 12, 13, 14, 15 connects the internal volume V11 to the outside of the body 10 via a passage which passes transversely right through the barrel 11 and extends into the corresponding port in a longitudinal direction thereof. Each port 12, 13, 14, 15 forms a connector enabling the body 10 to be coupled to a suitable medical hardware. In the example envisioned in the figures, the ports 12, 13 and 14 respectively form female connectors which in this case are each provided with an external screw thread 16 able, by screw-fastening, to accept for example a Luer fitting; the port 15 for its part forms a male connector, preferably a Luer connector, which in this case is equipped with a locking ring 17. Be that as it may, the specifics of the ports 12 to 15 as relates to their capacity for coupling to medical hardware that can be used with the medical valve 1 are nonlimiting. Likewise, each of the ports 12 to 15 may be used indiscriminately as an inlet port or as an outlet port, depending on the context in which the medical valve 1 is being used.

The plug 20, which is depicted in isolation in FIGS. 3 to 5, comprises a barrel 21. As is clearly visible in FIGS. 4 and 5, the barrel 21 is tubular, and centered on a geometric axis Z21.

When the medical valve 1 is in the assembled state, the barrel 21 is mounted on the body 10, being at least partially housed inside the internal volume V11, while being able to be rotated therein about the axis Z21 with respect to the body 10. To this end, the barrel 21 includes a tubular wall 22 which, when the medical valve 1 is in the assembled state, is fully housed inside the internal volume V11 and which, in the exemplary embodiment considered in the figures, forms the running part of the barrel 21 along the axis Z21. In addition, according to an advantageous arrangement, the barrel 21 is, at one of its two terminal parts 21A and 21B which are axially opposite one another along the axis Z21, provided with a means for grasping 23, in this instance a lever, so that a user can grasp it manually in order to rotate the plug 20 about the axis Z21 with respect to the body 10. Furthermore, according to a likewise-advantageous arrangement, the barrel 21 is, at its terminal part 21B opposite its terminal part 21A that is provided with the means for grasping 23, provided with a connecting and retaining means 24, in this instance a flange, to hold the barrel 21 inside the internal volume V11 and fixedly connect the plug 20 and the body 10 to one another along the axis Z21, for example by clip-fastening or heading. The tubular wall 22 connects the terminal parts 21A and 21B of the barrel 21 to one another, extending from one axial end 22A of this tubular wall 22 to an axial end 22B of this tubular wall 22 that is the opposite end from the axial end 22A along the axis Z21, these axial ends 22A and 22B facing respectively toward the terminal part 21A and the terminal part 21B of the barrel 21.

The tubular wall 22 is provided with an exterior surface 22C which is designed to complement the internal volume V11 so that when the medical valve 1 is in the assembled state, the mounting of the barrel 21 inside the internal volume V11 is fluidtight. About the axis Z21, this exterior surface 22C extends over 360°. Along the axis Z21, this exterior surface 22C extends over the entire axial extent of the tubular wall 22, or in other words from one of the two axial ends 22A and 22B to the other. In practice, this exterior surface 22C is cylindrical and/or slightly frustoconical, being centered on the axis Z21, the corresponding specifics of this exterior surface 22C being nonlimiting, provided that the collaboration between this exterior surface 22C and the internal volume V11 via the complementing shapes thereof renders the rotary mounting of the barrel 21 inside this internal volume V11 fluidtight. In particular, the exterior surface 22C of the tubular wall 22 advantageously exhibits, in any geometric plane perpendicular Z21, a circular contour which is centered on this axis Z21, as clearly visible in inserts A), B) and C) of FIG. 5.

With regard to its tubular shape, the tubular wall 22 is also provided with an interior surface 22D which, unlike the exterior surface 22C, faces radially toward the axis Z21. About the axis Z21, the interior surface 22D extends over 360°. Along the axis Z21, the interior surface 22D extends over the entire axial extent of the tubular wall 22.

The plug 20 also comprises a passage 25 which is indissociable from the barrel 21 and which, depending on the angular position of the barrel 21 about the axis Z21 with respect to the body 10, establishes communication between at least two of the ports 12 to 15 of the body 10. In other words, the passage 25 controls the establishment of communication between the ports 12 to 15 according to the angular position of the plug 20 about the axis Z21 with respect to the body 10: thus, when the barrel 21 occupies an ad hoc angular position, the passage 25 allows a flow to pass through it between at least two of the ports 12 to 15, while isolating the other port or ports, if any; and when the barrel 21 occupies some other ad hoc angular position, the passage 25 allows a flow to pass through it between at least another two of the ports 12 to 15, while isolating the other port or ports, if any. For example, FIG. 2 clearly shows that, in the angular position of the barrel 21 which is being considered in this FIG. 2, the passage 25 establishes communication between the ports 12 and 13, while isolating the ports 14 and 15.

The passage 25 passes all the way through the tubular wall 22 of the barrel 21, which is to say from the exterior surface 22C to the interior surface 22D of the tubular wall 22, and extends inside the barrel 21 where the passage 25 is delimited by a passage wall 26 projecting from the interior surface 22D. The passage 25 opens onto the exterior surface 22C via at least two distinct openings which are connected to one another by the passage 25 and which are distributed about the axis Z21, all of the openings being situated in the one same portion 22.1 of the tubular wall 22, this portion 22.1 extending about the axis Z21 over at most approximately 180° from one of these openings to another of these openings, as indicated schematically in insert B) of FIG. 5. Depending on the angular position of the barrel 21 about the axis Z21 with respect to the body 10, the aforementioned openings can be aligned with an equivalent number of respective ports from among the ports 12 to 15 of the body 10. In the embodiment considered in FIGS. 1 to 5, there are two aforementioned openings, referenced 25A and 25B respectively, so that the passage 25 is advantageously able to establish communication between only two of the ports 12 to 15, while isolating the other two ports, depending on the angular position of the barrel 21. In addition, again in the embodiment considered in FIGS. 1 to 5, the portion 22.1 of the tubular wall 22 extends advantageously over approximately 90° about the axis Z21, as illustrated schematically in inserts A), B) and C) of FIG. 5. The passage 25 is then advantageously L-shaped, as is clearly visible in FIG. 2 and in insert B) of FIG. 5. In all cases, the openings of the passage 25, such as the openings 25A and 25B, are all situated, along the axis Z21, both between and some distance away from the axial ends 22A and 22B of the tubular wall 22; in the embodiment considered in the figures, the openings 25A and 25B are thus situated axially midway between the axial ends 22A and 22B.

The passage wall 26 extends, at least in part, from the portion 22.1 of the tubular wall 22, as is clearly visible in FIG. 4 and in insert B) of FIG. 5. In the embodiment considered here, the passage wall 26 thus extends from the portion 22.1 of the tubular wall 22, projecting from the interior surface 22D of the tubular wall 22 in a direction that is radial with respect to the axis Z21.

Notably with a view to stiffening the structure formed by the passage wall 26 inside the barrel 21, the plug 20 advantageously comprises a partition 27 which, as is clearly visible in FIG. 4, extends perpendicular to the axis Z21 and connects the passage wall 26 and a portion 22.2 of the tubular wall, diametrically opposite the portion 22.1.

Before describing certain dimensional aspects of the plug 20 in greater detail, it should be noted that the barrel 21, the passage 25 and the partition 27 are advantageously produced as a single piece, in this instance forming the entirety of the plug 20, which is made of a plastics material. This plastics material is notably shaped by injection molding. In any case, the plastics material of which the plug 20 is made advantageously allows this plug to conform, through slight deformation, to the internal volume V11 of the body 10. The plastics material of the plug 20 is preferably selected from among polyethylene (PE), polypropylene (PP), polyoxymethylene (POM) and polybutylene terephthalate (PBT), more preferentially still being selected as being made of polyoxymethylene (POM).

Moreover, the plastics material of the plug 20 is advantageously different from the material of which the barrel 11 and, more generally, the body 10 are made, notably so as to improve the properties of rotation of the plug 20 in the body 10. In particular, the barrel 11 is advantageously made from a plastics material numerous examples of which are given in WO2016/166346, to which the reader may refer. The barrel 11 is preferably made of polyamide or from a polyamide-containing plastics material.

In any case, the materials of which the body 10 and the plug 20 are made are able to withstand the mechanical and chemical stresses to which the medical valve 1 is intended to be subjected. The mechanical stresses are essentially deformation in shear and the pressure exerted on the medical valve 1 during its manufacture and its use. The chemical stresses are essentially associated with the products intended to circulate inside the medical valve 1: in practice, the aforementioned materials are resistant to any pharmaceutical product, including oily products, particularly the product Lipiodol®.

Returning now to the description of certain dimensional aspects of the plug 20, FIGS. 4 and 5 clearly show that the tubular wall 22 does not have a constant thickness about the axis Z21. More specifically, the tubular wall 22 is internally thicker in its portion 22.1 than in the rest of the tubular wall 22. This amounts to stating that the tubular wall 22 is provided with an internal thickening, located in its portion 22.1, in comparison with the rest of the tubular wall 22. Along the axis Z21, this internal thickening extends on each side of the passage 25 or even, as here, over the entire axial extent of the tubular wall 22. As a result, as is clearly visible in inserts A) and C) of FIG. 5, the interior surface 22D of the tubular wall 22 exhibits, axially on each side of the passage 25, a transverse contour, which is to say a contour in any plane perpendicular to the axis Z21, which is off-centered with respect to the axis Z21.

Thanks to the internal thickening of the tubular wall 22 of the barrel 21, which thickening is located in the portion 22.1 of this tubular wall 22, the resistance of the medical valve 1 to leakage is substantially enhanced. Without wishing to be bound by a theory, the inventors have discovered that this localized internal thickening causes a tendency for the contact pressure at the interface between the tubular wall 22 and the internal volume V11 of the barrel 11 to be increased specifically in the portion 22.1 of the tubular wall 22, and for this to be the case axially on each side of the passage 25, thus forming respectively two sealing barriers controlling seepage at the aforementioned interface and thereby minimizing leakage to the outside of this interface when the medical valve 1 is being used, including at high usage pressures typically greater than 65 bar. The enhanced resistance to leakage is thus localized to that zone of the plug 20 on which the greatest pressure is exerted when the medical valve 1 is in use, while at the same time spreading the pressure field to the diametrically opposite zone of the plug 20. At the same time, the torque needed to rotate the plug 20 with respect to the body 10 about the axis Z21 is not significantly affected as a result.

Hereinafter, the term “barrel thickness” is used to describe the dimension of the tubular wall 22 which is radial with respect to the axis Z21 and which separates the exterior surface 22C and interior surface 22D of this tubular wall 22 from one another. As is clearly visible in FIGS. 4 and 5, in which the barrel thickness is referenced E, this barrel thickness E varies about the axis Z21, advantageously continuously, being greater in the portion 22.1 than in the rest of the tubular wall 22. Thus, in FIG. 4, the barrel thickness E exhibits different respective values in the left-hand half and in the right-hand half of this figure. It should be noted that, depending on the geometric specifics of the exterior surface 22C and interior surface 22D of the tubular wall 22, the barrel thickness E may, at a given point on the periphery of the tubular wall 22 and whatever the position of that point about the axis Z21, not be rigorously constant over the entire axial extent of the tubular wall 22; however, in a given plane perpendicular to the axis Z21, and whatever the position of this plane over the entire axial extent of the tubular wall 22, the barrel thickness E is greater in the portion 22.1 than in the rest of the tubular wall 22, as is clearly visible in FIG. 4.

In the embodiment considered in FIGS. 1 to 5, the barrel thickness E varies about the axis Z21, advantageously continuously, between a maximum Emax, which is reached at a first point on the periphery of the tubular wall 22, this point being situated in the portion 22.1 and midway between the openings 25A and 25B about the axis Z21, and a minimum Emin, which is reached at a second point on the periphery of the tubular wall 22, diametrically opposite the aforementioned first point, as indicated in FIG. 5.

According to optional arrangements, which are implemented in the embodiment considered in FIGS. 1 to 5 and which are aimed at further enhancing the resistance to leakage of the medical valve 1, the passage wall 26 and the partition 27 exhibit specific dimensions, detailed hereinafter.

The term “passage thickness” is used to describe the axial dimension of the passage wall 26 axially on each side of the passage 25. This passage thickness is referenced F in FIG. 4. The term “partition thickness” is used to describe the axial dimension of the partition 27. This partition thickness is referenced G in FIG. 4.

In a first advantageous arrangement, the passage thickness F is less than the minimum value of the barrel thickness E in the portion 22.1 of the tubular wall 22, and the partition thickness G is less than the minimum value of the barrel thickness E in the portion 22.2 of the tubular wall 22. This then avoids shrinkage-cavity phenomena during the manufacture of the plug 20. Of course, the passage thickness F and the partition thickness G need to exhibit a minimum value associated with the fact that the plastics material can fill the corresponding molding cavities without the risk of a shortage of material.

According to another advantageous arrangement, the partition thickness G is greater than the passage thickness F. This then further limits the deformation of the passage wall 26 perpendicular to the axis Z21 when the passage 25 is pressurized, namely when a fluid under high pressure, typically greater than 65 bar, is flowing in the passage 25.

In yet another advantageous arrangement, the passage thickness F is equal to 40%, plus or minus 10%, of the minimum value of the barrel thickness E in the portion 22.1 of the tubular wall, and the partition thickness G is equal to 85%, plus or minus 10%, of the minimum value of the barrel thickness E in the portion 22.2 of the tubular wall 22. In this way, the inventors have established that the deformations of the passage 25 under pressure are controlled, avoiding both significant deformation of the passage wall 26 in the direction transverse to the axis Z21 and significant deformation of the edges of the openings 25A and 25B at the interface between the tubular wall 22 and the internal volume V11 of the barrel 11.

As mentioned above, the openings via which the passage 25 opens onto the exterior surface 22C of the tubular wall 22 of the barrel 21, such as the openings 25A and 25B, may be provided in a quantity greater than two. Likewise, the angular extent of the portion 22.1 of the tubular wall 22 is not restricted to approximately 90° provided that this angular extent remains less than approximately 180°. As a result, rather than being L-shaped, the passage 25 may exhibit numerous other geometric shapes. By way of example, this aspect is illustrated in FIG. 6 which shows an alternative embodiment for the plug 20, referenced 20′.

The plug 20′ is functionally similar to the plug 20 while having structural differences therefrom, as detailed hereinafter. The plug 20′ thus comprises, on the one hand, a barrel 21′, which is functionally similar to the barrel 21, notably being centered on a geometric axis Z21′ functionally similar to the axis Z21, and including a tubular wall 22′ functionally similar to the tubular wall 22 and, on the other hand, a passage 25′ which is functionally similar to the passage 25 but which structurally differs therefrom in that the passage 25′ opens onto the exterior surface 22C′ of the tubular wall 22′ not via two openings but via three openings which are respectively referenced 25A′, 25B′ and 25C′ as is clearly visible in insert B) of FIG. 6. The openings 25A′, 25B′ and 25C′ are connected to one another by the passage 25′ and are distributed about the axis Z21′ while all being situated in the one same portion 22.1′ of the tubular wall 22′. The portion 22.1′ of the tubular wall 22′ is functionally similar to the portion 22.1 of the tubular wall 22, but structurally differs therefrom in that the portion 22.1′ extends over approximately 180°. In this case, the openings 25A′, 25B′ and 25C′ are distributed about the axis Z21′ substantially uniformly, so that the passage 25′ is T-shaped, as is clearly visible in insert B) of FIG. 6.

In accordance with considerations similar to those detailed hereinabove in respect of the plug 20, the tubular wall 22′ of the barrel 21′ of the plug 20′ is internally thicker in its portion 22.1′ than in the rest of the tubular wall 22′, as is clearly visible in FIG. 6. The barrel thickness, denoted E′, of the barrel 21′ thus varies about the axis Z21′, advantageously continuously, being greater in the portion 22.1′ than in the rest of the tubular wall 22′.

In the embodiment of FIG. 6, the barrel thickness E′ thus varies about the axis Z21′, advantageously continuously, between:
a maximum Emax′ which is reached and substantially maintained over the entirety of the portion 22.1′ about the axis Z21′, and
a minimum Emin′ which is reached at a point on the periphery of the tubular wall 22′, this point being situated, about the axis Z21′, substantially in the middle of a portion 22.2′ of the tubular wall 22′ that is the complement of the portion 22.1′ thereof, as is clearly visible in inserts A) and C) of FIG. 6.

As a result, as is clearly visible in inserts A) and C) of FIG. 6, the interior surface 22D′ of the tubular wall 22′ exhibits, axially on each side of the passage 25′, an egg-shaped transverse contour oriented toward that point on the periphery of the tubular wall 22′ that is associated with the minimum Emin′.

Moreover, although not reprised in detail here, the optional considerations detailed hereinabove in respect of the plug 20 in connection with the passage wall 26 and the partition 27 apply, mutatis mutandis, to the plug 20′.

Finally, various arrangements and variants to the medical valve 1 described hitherto are also conceivable. By way of examples:
the amplitude of the rotational movement of the plug 20 or 20′ with respect to the body 10 may be restricted by ad hoc arrangements of the medical valve 1 so as to prevent the establishment of communication between certain of the ports 12 to 15, as detailed for example in WO 2016/166346, to which the reader may refer; the medical valve 1 provided with the four ports 12 to 15 may thus notably be designed as an only three-way valve; and/or
the number of ports, such as the ports 12 to 15, is not restricted to four but may be equal to three or else equal to five or more, each of these various ports being able indiscriminately to form either a female connector or a male connector.

1. A medical valve, comprising:
a body, which defines an internal volume and which is provided with at least three ports each of which connects the internal volume with the outside of the body, and
a plug which comprises:
a barrel, which is tubular, centered on an axis, and which includes a tubular wall provided with an exterior surface which complements the internal volume so that the barrel is housed in a fluidtight manner inside the internal volume while being able to be rotated therein about the axis with respect to the body, and
a passage that establishes communication between at least two of the ports depending on the angular position of the barrel about the axis with respect to the body, which passage passes through the tubular wall, from the exterior surface to an interior surface of the tubular wall, and extends inside the barrel in such a way as to connect to one another at least two openings via which the passage opens onto the exterior surface of the tubular wall, these openings being distinct from one another and all situated in the one same first portion of the tubular wall, which extends about the axis over more than 180° from one of the openings to another of the openings,
characterized in that the tubular wall is, axially on each side of the passage, internally thicker in its first portion than it is in the rest of the tubular wall.

2. The medical valve as claimed in claim 1, wherein only two openings are provided.

3. The medical valve as claimed in claim 1, wherein the first portion of the tubular wall extends about the axis over 90°.

4. The medical valve as claimed in claim 2, wherein the first portion of the tubular wall extends about the axis over 90°, and wherein the passage is L-shaped.

5. The medical valve as claimed in claim 1, wherein three openings are provided.

6. The medical valve as claimed in claim 1,
wherein the first portion of the tubular wall extends about the axis over 180°.

7. The medical valve as claimed in claim 1,
wherein the exterior surface of the tubular wall exhibits, in any plane perpendicular to the axis, a circular contour which is centered on the axis,
and wherein the tubular wall exhibits a dimension, which is radial with respect to the axis and referred to as the barrel thickness, which separates the exterior surface and interior surface of the tubular wall from one another and which varies about the axis, being greater in the first portion than in the rest of the tubular wall.

8. The medical valve as claimed in claim 2,
wherein the exterior surface of the tubular wall exhibits, in any plane perpendicular to the axis, a circular contour which is centered on the axis,
wherein the tubular wall exhibits a dimension, which is radial with respect to the axis and referred to as the barrel thickness, which separates the exterior surface and interior surface of the tubular wall from one another and which varies about the axis, being greater in the first portion than in the rest of the tubular wall, and wherein the barrel thickness varies about the axis between:
a maximum which is reached at a first point on the periphery of the tubular wall, this point being situated in the first portion and midway between the two openings about the axis, and
a minimum which is reached at a second point on the periphery of the tubular wall, diametrically opposite the first point.

9. The medical valve as claimed in claim 5,
wherein the exterior surface of the tubular wall exhibits, in any plane perpendicular to the axis, a circular contour which is centered on the axis,
wherein the tubular wall exhibits a dimension, which is radial with respect to the axis and referred to as the barrel thickness, which separates the exterior surface and interior surface of the tubular wall from one another and which varies about the axis, being greater in the first portion than in the rest of the tubular wall, and wherein the barrel thickness varies about the axis between:
a maximum which is reached and maintained over the entirety of the first portion about the axis, and
a minimum which is reached at a point on the periphery of the tubular wall, this point being situated, about the axis, in the middle of a portion of the tubular wall that is the complement of the first portion.

10. The medical valve as claimed in claim 7,
wherein the barrel thickness varies about the axis continuously.

11. The medical valve as claimed in claim 7,
wherein the passage is, inside the barrel, delimited by a passage wall which extends from the first portion of the tubular wall, so that it projects radially from the interior surface of the tubular wall,
wherein the passage wall exhibits, axially on each side of the passage, an axial dimension, referred to as the passage thickness, which is less than the minimum value of the barrel thickness in the first portion of the tubular wall,
wherein the plug also has a partition which extends perpendicular to the axis and which connects the passage wall and a second portion of the tubular wall which is diametrically opposite the first portion of the tubular wall,
and wherein the partition exhibits an axial dimension, referred to as the partition thickness, which is less than the minimum value of the barrel thickness in the second portion of the tubular wall.

12. The medical valve as claimed in claim 11, wherein the partition thickness is greater than the passage thickness.

13. The medical valve as claimed in claim 11,
wherein the passage thickness is equal to 40%, plus or minus 10%, of the minimum value of the barrel thickness in the first portion of the tubular wall,
and wherein the partition thickness is equal to 85%, plus or minus 10%, of the minimum value of the barrel thickness in the second portion of the tubular wall.

14. The medical valve as claimed in claim 6, wherein three openings are provided.

15. The medical valve as claimed in claim 8, wherein the first portion of the tubular wall extends about the axis over 90°, and wherein the passage is L-shaped.
